(19) United States
(12) Patent Application Publication
(10) Pub. No.: US 2010/0105022 A1
(43) Pub. Date: Apr. 29, 2010

(54) ANALYZING BIOLOGICAL CELL MATERIAL BASED ON DIFFERENT INTERACTIONS BETWEEN ILLUMINATION LIGHT AND CELL COMPONENTS

(75) Inventors: Stein Kuiper, Vught (NL); Bernardus Hendrikus Wilhelmus Hendriks, Eindhoven (NL); Ruth Wilhelmine Ireen De Boer, Den Bosch (NL); Gert Hooft, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/530,787

(22) PCT Filed: Mar. 10, 2008
(86) PCT No.: PCT/IB08/50856

§ 371 Date: Sep. 11, 2009

(30) Foreign Application Priority Data
Mar. 13, 2007 (EP) 07103980.4

(51) Int. Cl. C12Q 1/00; C12M 1/34

(52) U.S. Cl. 435/4; 435/288.7; 435/287.2

(57) ABSTRACT

It is described a device (100) for analyzing biological cell material (115). The device (100) comprises a light source arrangement (120), which is adapted for directing a first (131) and a second illumination light (132) towards the cell material (115), wherein the first (131) and the second (132) illumination light comprises a first and a second spectral radiation component, respectively. The device (100) further comprises a detector arrangement (170), which is adapted for receiving a first measurement light (151) based on a first interaction of the first illumination light (131) with the cell material (115) and a second measurement light (152) based on a second interaction of the second illumination light (152) with the cell material (115). Further, the device (100) comprises an evaluation unit (180), which is coupled the detector arrangement (170) and which is adapted to evaluate a first signal (171a) and a second signal (171b) being indicative for the first (151) and the second measurement light (151), respectively. The device (100) may be used for accomplishing ultraviolet DNA image cytometryin combination with autofluorescence measurements of NAD(P)H.

### FIELD OF INVENTION

The present invention relates to a device and to a method for analyzing biological cell material for instance for the purpose of distinguishing cancerous tumor cells from benign tumor cells.

### ART BACKGROUND

Early detection of tumors is the most important prerequisite for fighting them effectively. This will also decisively improve tumor patient's prospects of being cured. In order to avoid multiple surgeries it is desirable to identify tumor tissue during one single. For further removing tissue from the patient the surgeon can than take into account reliable information about the type of tumor cells.

At present, tumor border demarcation is performed by analysis of so-called ‘frozen sections’ by a pathologist. This technique takes approximately one hour; often, no intra-operative investigation is performed because of the long waiting time during which the patient lies open on the operation table. An alternative is to perform DNA cytometry for tumor border demarcation, on the condition that the result is quickly available. However, the selection of suspicious and normal cells by a pathologist is a time consuming and therefore an expensive procedure. A pathologist typically takes half an hour to select enough cells for a statistically reliable result. Moreover, the standard Feulgen staining procedure for DNA cytometry takes five hours.

US 2002/0128557 A1 discloses an apparatus for detecting tumorous tissue comprising at least one excitation light source, which first excitation light source emits a first excitation light of a wavelength of between 300 nm and 314 nm and includes at least one optical fiber for guiding the first excitation light to an object field of the tissue to be examined. The apparatus further comprises at least one lens for projecting an autofluorescence signal and/or a remission signal of the tissue, generated by the first excitation light, to a CCD or ICCD chip of a camera. Further, the apparatus comprises a data processing system for processing the signals transmitted by the camera. The lens is capable of processing UV light and being designed such that at least two images from different spectral regions of the fluorescent object field are generated and projected to the CCD or ICCD chip. At least one image represents the UV range and another different wavelength range of the auto-fluorescence signal and/or of the remission signal of said object field.

U.S. Pat. No. 5,131,398 discloses a method and apparatus for distinguishing cancerous tumors and tissue from benign tumors and tissue or normal tissue using native fluorescence. The tissue to be examined is excited with a beam of monochromatic light at 300 nm. The intensity of the native fluorescence emitted from tissue is measured at 340 nm and 440 nm. The ratio of the two intensities is then calculated and used as a basis for determining if the tissue is cancerous as opposed to benign or normal. The method and the apparatus rely on the discovery that when tissue is excited with monochromatic light at 300 nm, the native fluorescence spectrum over the region from about 320 nm to 600 nm is the tissue that is cancerous and substantially different from the native fluorescence spectrum that would result if the tissue is either benign or normal. The technique is useful for in-vivo and in-vitro testing of human as well as animal tissue.

There may be a need for providing a device and a method, which allows both for a quick and reliable analysis of biological cell material.

### SUMMARY OF THE INVENTION

This need may be met by the subject matter according to the independent claims. Advantageous embodiments of the present invention are described by the dependent claims.

According to a first aspect of the invention there is provided a device for analyzing biological cell material. The described device comprises (a) a light source arrangement, which is adapted for directing a first illumination light and a second illumination light towards the cell material, wherein the first illumination light comprises a first spectral radiation component and the second illumination light comprises a second spectral radiation component, and (b) a detector arrangement, which is adapted for receiving a first measurement light, which is based on a first interaction of the first illumination light with the cell material, and a second measurement light, which is based on a second interaction of the second illumination light with the cell material. The described biological cell material analyzing device further comprises (c) an evaluation unit, which is coupled the detector arrangement and which is adapted to evaluate a first signal being indicative for the first measurement light and a second signal being indicative for the second measurement light.

This aspect of the invention is based on the idea that in many applications different indicators for a disease, which modifies the structure and/or the composition at least of some cells, are independent from each other. Therefore, by combining at least two independent measurements being indicative for different properties of the cell material the diagnostic accuracy can be significantly increased. This holds in particular for the identification of cancer cells.

The combined analysis of the cell material may make a selection of suspicious and normal cells by a pathologist no more necessary. Therefore, a time consuming and expensive cell sorting procedure before the real cytometry measurement may become obsolete. This may provide the advantage that tissue having been withdrawn from a patient can be analyzed much faster and also the result of the diagnostic procedure can be obtained much faster. Therefore, the time span, during which a patient lies open on an operation table, and as a consequence the risk for infections, can be reduced significantly.

It has to be mentioned that in this application the term light is used for electromagnetic radiation comprising the ultraviolet, the visible and the infrared spectral range. Therefore, the first illumination light, the second illumination light, the first measurement light and the second measurement light can have any wavelength within this spectral range.

It has to be mentioned that the described cell analyzing device may be used both for in-vivo and in-vitro applications. In case of an in-vivo application the tissue of a patient can be directly analyzed by directing the first and the second illumination light directly onto the tissue under study.

According to an embodiment of the invention the device further comprises a carrier element for supporting the cell material. The carrier element may be any element, which is suitable for keeping the biological cell material within the light path of the first respectively the second measurement light. The carrier element may be for instance an object holder, which is well known from microscopy. However, the carrier element may also be a tube for guiding the biological cell material as it passes the light path of the first respectively the second measurement light. Such a tube is well known from flow cytometry apparatuses such as for instance fluorescent-activated cell sorting (FACS) instruments.

According to a further embodiment of the invention the first interaction is absorption and/or the second interaction is fluorescence. This may provide the advantage that the device can be realized by means of comparatively simple optical setups.

In this respect it has to be mentioned that in this application the term absorption is used not only for electromagnetic radiation respectively photons being physically absorbed within the biological cell material under study. The term absorption is supposed to rather cover all physical interactions, which reduce the intensity of a light beam penetrating the cell material. Such an intensity reducing interaction is for instance light scattering, which causes scattered radiation to be removed from the primary light beam. Of course, instead of measuring an intensity reduction of the transmitted light beam the strength of the first interaction can also be acquired by measuring an intensity of sideways scattered light.

At this point it is mentioned that of course the second measurement light, which is caused by the described fluorescence interaction, is emitted into a solid angle of 4π. This means that the second measurement light can be detected in any direction with respect to the direction of the second illumination light.

According to a further embodiment of the invention (a) the first illumination light is adapted to interact with a first cell component of the biological cell material by means of the first interaction and/or (b) the second illumination light is adapted to interact with a second cell component of the biological cell material by means of the second interaction. This means that the described biological cell analyzing device may be optimized for independently analyzing different measurement values, which are indicative for properties of different cell components. Since many diseases such as cancer cause different modifications to pathogenic cells, the described device allows for analyzing biological cells with a high accuracy. Therefore, the reliability for identifying and/or evaluating the amount of pathogenic cells can be increased significantly.

According to a further embodiment of the invention the first cell component is DNA and/or the second cell component is an enzyme being used for cell metabolism. In particular the second cell component is NAD(P)H. This is based on the observation that a first indicator for cancer being an increased amount of DNA in the nucleus and a second indicator for cancer being an increased level of NAD(P)H are substantially independent. This means that two different cell components can be simultaneously investigated, which both have been found to be highly indicative for the identification of malign cancer cells.

In particular, the degree of absorption can be highly indicative for the amount of DNA material being present within the cell material. Since malign cells typically comprise a higher amount of DNA, the absorption caused by the biological cell material may be used as a first indicator for classifying the cells into benign cells or malignant cells. For an overview of DNA absorption measurements reference is made to “G. Haroske, F. Giroud, A. Reith and A. Rocking, 1997 ESACP consensus report on diagnostic DNA image cytometry, Analytical Cellular Pathology 17 (1998) 189-200”. The disclosure of this publication is hereby incorporated by reference.

In order to increase the sensitivity for detecting the first interaction the DNA can be stained with appropriate fluorescence molecules. For staining the DNA in particular the known method of Feulgen is considered as to be appropriate.

With respect to NAD(P)H representing the second cell component it has to be mentioned that, when the second illumination light comprises an appropriate spectral component for exciting NAD(P)H, the strength of the corresponding fluorescence signal will be highly indicative for the concentration of NAD(P)H within the cells under study. Since in cancer cells this concentration is much higher than in normal cells, the intensity of the second measurement light is also indicative for classifying the cells into benign cells or malignant cells. For details regarding different concentrations of NAD(P)H within different types of cells, reference is made to the publication “I. Georgakoudi et al., NAD(P)H and collagen as in vivo quantitative fluorescent biomarkers of epithelial precancerous changes, Cancer Research 62 (2002) pp. 682-687”. The disclosure of this publication is hereby incorporated by reference.

The described combined evaluation of both the amount of DNA and the concentration of NAD(P)H within the cells under study is much more reliable for identifying cancer cells than a procedure relying solely on the NAD(P)H concentration. This is the case because it may happen that some normal cells may also show an increased NAD(P)H concentration due to an occasionally increased cell activity (e.g. proliferating cells). As a consequence, the described cell analyzing device may provide the advantage that the reliability for identifying cancer cells is significantly increased as compared to known apparatuses as for instance FACS instruments.

According to a further embodiment of the invention the first illumination light and the second illumination light is ultraviolet light comprising a wavelength in between 150 nm and 350 nm, preferably in between 200 nm and 300 nm and more preferably in between 230 nm and 270 nm.

This may provide the advantage that the amount of the first cell component respectively the amount of DNA within cells can be detected without performing an elaborate staining procedure, like for example the Feulgen staining procedure. This is based on the fact that DNA absorbs ultraviolet light of the described spectral ranges around 260 nm much stronger than other cell components. Therefore, by omitting cell staining the described device allows for a much faster and cheaper DNA-cytometry.

According to a further embodiment of the invention the second illumination light is ultraviolet light comprising a wavelength in between 280 nm and 400 nm, preferably in between 320 nm and 360 nm and more preferably in between 330 nm and 350 nm. Further, the second measurement light is visible light comprising a wavelength in between 345 nm and 545 nm, preferably in between 400 nm and 490 nm and more preferably in between 430 nm and 460 nm.

This may provide the advantage that the concentration of the second cell component respectively the concentration of NAD(P)H can be detected without performing an elaborate staining procedure. This is based on the fact that NAD(P)H itself can be excited by ultraviolet light. Thereby, the ultraviolet light comprises spectral components within the described spectral ranges around 340 nm. A subsequent de-excitation generates a fluorescence light respectively a second measurement light within spectral ranges around 445 nm. Since no staining respectively marking of NAD(P)H with fluorescence molecules is necessary, the physical effect of exciting and de-exciting NAD(P)H are also described by the term autofluorescence.

According to a further embodiment of the invention the light source arrangement comprises a broad-spectrum ultraviolet light source. The light may be for instance a broad-spectrum UV lamp. Such a UV lamp may represent the only light source of the described cell analyzing device.

Appropriate spectral pass filter may by used, which, in the spectral domain, shape the light originating from the ultraviolet light source such that the first illumination light and the second illumination light can be spectrally distinguished from each other. However, a spectral overlap of the first illumination light and the second illumination light is possible.

A common spatial light path may be used both for the first and the second illumination light. This means that the spectral pass filter, which can be a single pass filter or an appropriate combination of at least two different spectral filters, exhibits two transmission maxima. A first transmission maximum, which may be optimized for the first interaction and a second transmission maximum, which may be optimized for the second interaction.

In case both the amount of DNA and the concentration of NAD(P)H within the cells under study is measured, in the spectral domain the first transmission maximum should be located around 260 nm (absorption maximum for DNA) and the second transmission maximum should be located around 340 nm (excitation maximum for NAD(P)H autofluorescence).

It has to be mentioned that in order to make an autofluorescence measurement of NAD(P)H more sensitive the spectral pass filter or a further blocking filter within the light path originating from the broad-spectrum ultraviolet light source should ensure that no visible light around 445 nm can hit the cell material under study. In this case it is guaranteed that all visible light respectively light around 445 nm, which may have been detected, has been generated by autofluorescence effects of NAD(P)H.

According to a further embodiment of the invention the light source arrangement comprises (a) a first light source generating the first illumination light and (b) a second light source generating the second illumination light.

In case both the amount of DNA and the concentration of NAD(P)H within the cells under study is measured, a deep UV-lamp such as a mercury lamp emitting a light spectrum containing a 253 nm line is appropriate for carrying out the above described DNA UV absorption measurement. For carrying out NAD(P)H autofluorescence measurements a 340 nm UV lamp such as a light emitting diode may be used preferably.

It has to be mentioned that also when using different light sources appropriate filter elements may be used. In particular, as has already been described above, a filter blocking visible light around 445 nm can be used for increasing the sensitivity of the NAD(P)H fluorescence measurements.

The first illumination light and the second illumination light can impinge onto the cell material under study at different angles of incidence. This may provide the advantage that the first measurement light being related with the first illumination light and the second measurement light being related with the second illumination light are spatially separated such that a separated detection of the first illumination light and the second illumination light can be realized simply by employing two corresponding detectors, which are arranged at different positions.

However, the first illumination light and the second illumination light can also impinge onto the cell material under study at the same angle of incidence. This means that before impinging the cell material the first illumination light and the second illumination light propagate along a common incidence beam path. For coupling both the light originating from the first light source and the light originating from the second light source with this common incidence beam path a beam splitter or preferably a dichroic mirror may be employed.

According to a further embodiment of the invention the device further comprises an optic arrangement for focusing the first illumination light and/or the second illumination light onto the biological cell material. This may provide the advantage that the biological cell material can be illuminated with a high spatial resolution such that even individual cells can be investigated.

The optic arrangement may comprise two lenses respectively optics, which are designed and arranged in such a manner that the first illumination light and/or the second illumination light propagates along an optical path corresponding to the optical path of a microscope. Thereby, an in particular high spatial resolution for illumination the biological material under study can be achieved.

In case the first illumination light and the second illumination light are optically coupled by means of a beam splitter and/or a dichroic mirror, one single optic arrangement can be used for both illumination lights.

According to a further embodiment of the invention the detector arrangement comprises (a) a first detector for receiving the first measurement light and (b) a second detector for receiving the second measurement light.

The first and/or the second detector may be for instance a camera comprising a spatial resolution. This may provide the advantage that also the light detection can be carried out with a spatial resolution. Thereby, at least one appropriate optic may be employed in order to provide for a spatial resolution, which allows for individually analyzing the cells under study.

In case both the detected first measurement light and the detected second measurement light originate from the biological cell material along the same direction, the first measurement light can be spatially separated from the second measurement light by means of a beam splitter or a dichroic mirror.

According to a further embodiment of the invention the detector arrangement comprises a common detector for receiving both the first measurement light and the second measurement light and the whole cell analyzing device further comprises a chopper device for letting pass the first measurement light and the second measurement light in an alternating manner.

Thereby, the chopper device may be equipped with (a) a first spectral filter element for letting pass the first measurement light and blocking the second measurement light and (b) a second spectral filter element for letting pass the second measurement light and blocking the first measurement light. This may provide the advantage that even when only a single common detector is used, the described cell analyzing device is adapted to individually measure the first measurement light and the second measurement light.

A further chopper device may be provided, which is arranged in the light path of the first illumination light respectively the second illumination light. The further chopper device may be equipped with (a) a further first spectral filter element for letting pass the first illumination light and blocking the second illumination light and (b) a further second spectral filter element for letting pass the second illumination light and blocking the first illumination light.

It has to be mentioned that also the common detector might be realized by means of a spatial resolving detector such as a camera. In particular in connection with an appropriate optic arrangement this may provide the advantage that the measurement light detection can be carried out with a spatial resolution, which allows for individually analyzing the cells under study.

According to a further embodiment of the invention (a) the first illumination light and the second illumination light impinge onto the biological cell material along a common incidence beam path and (b) the first measurement light and the second measurement light leave the biological cell material along a common exit beam path. Thereby, the common incidence beam path and the common exit beam path are collinear with respect to each other.

This may provide the advantage the described biological cell analyzing device can be realized with a comparatively simple optical setup comprising a single main optical axis. Preferably, this optical axis, which is defined both by the direction of the common incidence beam path and the direction of the common exit beam path, is oriented at least approximately perpendicular to a surface of the biological cell material.

According to a further embodiment of the invention (a) the detector arrangement is adapted to measure a time dependence of the second signal being indicative for the second measurement light and (b) the evaluation unit is adapted to evaluate the time dependence of the second signal. This may provide the advantage that a further discriminating property between healthy cells and cancer cells can be evaluated. Thereby, the reliability of a cell analyzing carried out with the described device can be further improved.

Preferably, the average fluorescence lifetime of the autofluorescence signal of NAD(P)H can be used. This possibility is based on the observation that in cancer cells the average autofluorescence lifetime of NAD(P)H is significantly shorter than in healthy cells. For more details regarding the fluorescence lifetime dependency on the type of cells reference is made to the publication “D. Elson et al., Time-domain fluorescence lifetime imaging applied to biological tissue, Photochem. Photobiol. Sci. 3, p. 795-801 (2004)”. The disclosure of this publication is hereby incorporated by reference.

According to a further embodiment of the invention the described biological cell material analyzing device further comprises a light modulation device, which is adapted to modulate the intensity of the second illumination light as a function of time.

This may provide the advantage, that a predefined time dependency of the second illumination light can be generated, which second illumination light effects the fluorescence excitation of for instance intracellular NAD(P)H. Therefore, the time dependency of the fluorescence de-excitation can be observed in a precise and easy manner just by measuring the time dependence of the second measurement light in a synchronized manner with respect to the modulation of the second illumination light. Thereby, the fluorescence de-excitation can be aligned in time with the fluorescence excitation.

The modulation device can be realized in many different manners. For instance the modulation device may be an appropriate electronic circuitry, which controls the light source emitting the second illumination light. Such a modulated control is in particular advantageous if the light source is a light emitting diode, which can be repeatedly switched on and off with a high repetition rate.

The modulation device might also be a chopper device, which repeatedly blocks at least the second illumination light. During the time spans, in which the second illumination light is blocked, no fluorescence excitation occurs and the fluorescence de-excitation of previously excited molecules can be observed. It is clear that the sensitivity for measuring the fluorescence lifetime is maximal, when the fluorescence excitation is carried out in a pulsed manner. This means that in between two fluorescence excitation time windows the second illumination light is suppressed completely. This holds of course both (a) for the embodiment wherein the light source emitting the second illumination light is controlled and (b) for the embodiment wherein a chopper device is used for modulating the second illumination light.

It has to be mentioned that absorption measurements carried out with the first illumination light do not show any time dependency because absorption and scattering effects always occur prompt at least with respect to the time scale of fluorescence de-excitation transitions. Therefore, provided that the time dependency of the first illumination beam is known, the absorption of the first illumination beam can be measured precisely just by comparing the intensity of the first measurement light with respect to the intensity of the first illumination light. This means that a modulation also of the first illumination beam does not cause absorption measurements of the first illumination beam to be less reliable.

According to a further aspect of the invention there is provided a method for analyzing biological cell material. The described method comprises (a) directing a first illumination light comprising a first spectral radiation component from a light source arrangement towards the cell material, (b) directing a second illumination light comprising a second spectral radiation component from the light source arrangement towards the cell material, (c) receiving a first measurement light, which is based on a first interaction of the first illumination light with the cell material, by means of a detector arrangement, and (d) receiving a second measurement light, which is based on a second interaction of the second illumination light with the cell material, by means of the detector arrangement. The described biological cell material analyzing method further comprises (e) evaluating a first signal being indicative for the first measurement light, and (f) evaluating a second signal being indicative for the second measurement light.

This aspect of the invention is based on the idea that by combining two independent measurement parameters, which are both indicative for a particular cell defect and/or a particular disease, the diagnostic accuracy for identifying the type of these cells can be significantly increased. This holds in particular for identifying cells as to be benign or malign because as compared to normal benign cells malign cancer cells show a comparatively strong change of optical properties in different spectral regimes. This change of optical properties may be based on structural and/or chemical changes of cancer cells as compared to normal cells.

The described combined analysis of the cell material under study may make a selection of suspicious and normal cells by a pathologist no more necessary. Therefore, a time consuming and expensive cell sorting procedure before the real cytometry measurement may become obsolete. This may provide the advantage that tissue having been withdrawn from a patient can be analyzed much faster and also the result of the diagnostic procedure can be obtained much faster. Therefore, the time span, during which a patient has to lie open on an operation table, can be reduced significantly.

It has to be mentioned that the described method can only be used with biological cell material, which has been irreversibly withdrawn from a patient's body. Therefore, when carrying out the described method there are no direct interactions with the living body of the patient. Further, the described method is not able to directly result in a diagnosis a patient is suffering. The described method can only help a physician to find a diagnosis based on his medical knowledge. Thereby, the physician can take into account also other diagnostic methods, which, in combination with the described method, can help the physician to make a diagnosis more reliable.

In other words, the described method is not used for providing a diagnosis or about treating patients. The described method and all other aspects and embodiments of the present inventing merely provides additional and more detailed information, which may assist a physician in reaching a diagnosis and/or in deciding about appropriate therapy procedures.

It has to be noted that embodiments of the invention have been described with reference to different subject matters. In particular, some embodiments have been described with reference to apparatus type claims whereas other embodiments have been described with reference to method type claims. However, a person skilled in the art will gather from the above and the following description that, unless other notified, in addition to any combination of features belonging to one type of subject matter also any combination between features relating to different subject matters, in particular between features of the apparatus type claims and features of the method type claims is considered to be disclosed with this application.

The aspects defined above and further aspects of the present invention are apparent from the examples of embodiment to be described hereinafter and are explained with reference to the examples of embodiment. The invention will be described in more detail hereinafter with reference to examples of embodiment but to which the invention is not limited.

### BRIEF DESCRIPTION OF THE DRAWINGS

All Figures show cell-analyzing devices for carrying out a UV DNA absorption cytometry measurements in combination with autofluorescence measurements of NAD(P)H.

FIG. 1 shows a cell-analyzing device comprising one UV broadband light source and two detectors.

FIG. 2 shows a cell-analyzing device comprising one UV broadband light source and one detector, wherein the spectral separation is carried out by means of chopper wheels.

FIG. 3 shows a cell-analyzing device comprising two UV light sources and one detector, wherein the spectral separation is carried out by means of chopper wheels.

FIG. 4 shows a cell-analyzing device for evaluating an autofluorescence lifetime of NAD(P)H, wherein the cell-analyzing device comprises two UV light sources and two detectors and wherein a modulation device is employed for modulating the intensity of an autofluorescence excitation light beam.

### DETAILED DESCRIPTION

The illustration in the drawing is schematic. It is noted that in different figures, identical elements or similar elements having the same function are provided with reference signs, which are different from the corresponding reference signs only within the first digit.

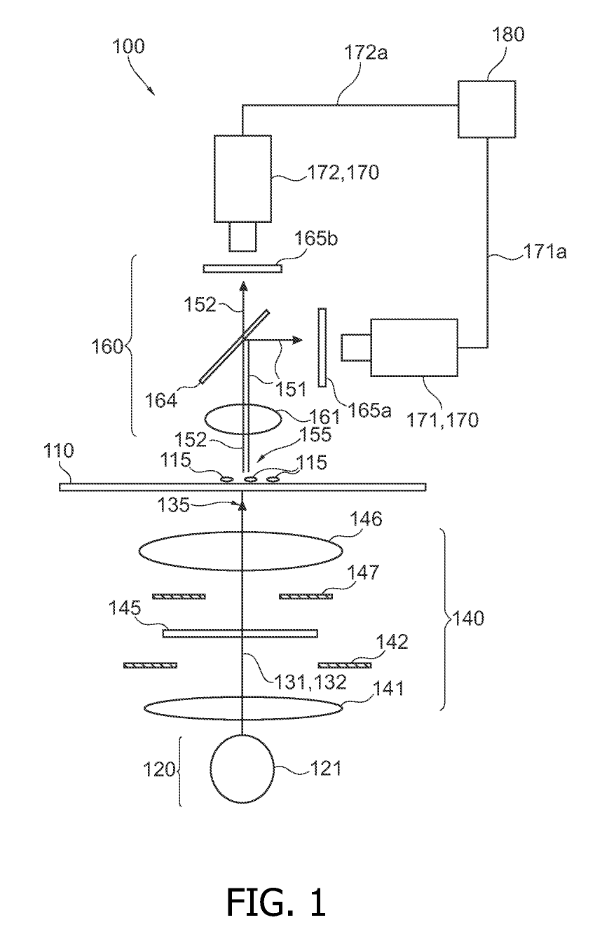

FIG. 1 shows a cell-analyzing device 100, which is adapted for carrying out a UV DNA absorption cytometry in combination with autofluorescence of NAD(P)H. The device comprises a carrier element 110, which is realized by means of an object holder known for instance from optical microscopy. The carrier element 110 supports cell material 115, which may contain both benign respectively normal cells and malign respectively cancer cells. The device 100 is adapted to facilitate an identification of the type of cells 115 such that compared to known devices for cell sorting both the speed and the reliability of the cell identification can be increased.

The device 100 comprises a light source arrangement 120, which according to the embodiment shown in FIG. 1 is a broad-spectrum ultraviolet light source 121. The light source 121 emits a broad band UV light beam, which is directed towards the carrier element 110. In between the light source 121 and the carrier element 110 there is arranged an optic arrangement 140. As can be seen from FIG. 1, the optic arrangement 140 comprises a field lens 141, a field iris 142, a condenser iris 147 and a condenser lens 146. All these optical elements are arranged in a symmetric manner with respect to the beam path of the UV light beam. In between the field iris 142 and the condenser iris 147 there is provided a spectral pass filter 145.

The spectral pass filter 145 is designed in such a manner that a first illumination light beam 131 having a spectral distribution around 260 nm and a second illumination light beam 132 having a spectral distribution around 340 nm can pass the spectral pass filter 145. Preferably, the spectral pass filter 145 comprises two transmission maxima around these wavelengths. However, also a spectral pass filter 145 having a single broad spectral transmission maximum such that both a first electromagnetic radiation having a wavelength distribution around 250 nm and a second electromagnetic radiation having a wavelength distribution around 340 nm can pass the filter 145.

According to the embodiment described here the field lens 141, the field iris 142, the condenser lens 146 and the condenser iris 147 represent a so-called Köhler illumination system. This may provide the advantage that a uniform illumination of the cell material 115 under study can be achieved, wherein an internal structure such as spiral-wound filament of the broad-spectrum ultraviolet light source 121 is not projected respectively imaged onto the plane in which the cell material 115 under study is present.

After having passed the filter 145 one can assume that the first illumination light 131 having a wavelength of around 250 nm and the second illumination light 132 having a wavelength of around 340 nm impinge onto the cell material 115. Thereby, both illumination light beams 131, 132 propagate along the same common incidence beam path 135.

The first illumination light 131 having a wavelength of around 260 nm will be absorbed predominately by the DNA within the nuclei of the cells 115 under study. Therefore, the amount of DNA within the cells 115 will determine the intensity of a first measurement light 152, which is transmitted through the cell material 115.

The second illumination light 132 having a wavelength of around 340 nm will predominately excite NAD(P)H such that upon a subsequent de-excitation of NAD(P)H an autofluorescence signal having a wavelength of around 445 nm can be observed. According to the embodiment described here this autofluorescence signal is observed along a direction being collinear to the direction of the first illumination light beam 131 respectively the second illumination light beam 132. The corresponding autofluorescence light beam representing a second measurement light is denominated with reference numeral 152. The second measurement light 152 and the first measurement light 151 leave the cell material 115 along a common exit beam path 155.

The cell-analyzing device 100 further comprises an optic arrangement 160. The optic arrangement 160 comprises an objective lens 161 and a dichroic mirror 164 being substantially reflective for radiation having a wavelength around 260 nm and substantially transparent for radiation having a wavelength around 445 nm. Therefore, the first measurement light 151, the intensity of which is indicative for the amount of DNA in the cell material 115, is reflected. Correspondingly, the second measurement light 152, the intensity of which is indicative for the concentration of NAD(P)H in the cell material 115, is transmitted.

The cell-analyzing device 100 further comprises a detector arrangement 170, which includes a first detector 171 and a second detector 172. The first detector 171 is equipped with a spectral pass filter 165a having a transmission maximum for wavelengths of around 260 nm. The second detector 172 is equipped with a pass filter 165b having a transmission maximum for wavelengths of around 445 nm. As can be seen from FIG. 1, the first detector 171 is spatially arranged for receiving the reflected first measurement light 151 whereas the second detector 172 is spatially arranged for receiving the transmitted second measurement light 152.

The first detector 171 provides a signal 171a, which is indicative for the first measurement light 151. The second detector 172 provides a signal 172a, which is indicative for the second measurement light 152. Both signals 171a and 172a are fed to an evaluation unit 180. The evaluation unit 180 is adapted to evaluate the two independent signals 171a and 172a by combining them in an appropriate way. Thereby, a further parameter may be generated, which may represent a reliable indicator for the type of cells being contained in the cell material 115. This further parameter can give a physician valuable information about the composition of the cell material 115.

According to the embodiment described here both the first detector 171 and the second detector 172 is realized by means of a camera. The cameras 171, 172 have a spatial resolution, which of course is also related with the focal length of the objective lens 161. The provision of a spatial resolution may provide the advantage that the light originating from individual cells 115 can be detected separately. As a consequence, the cells being contained in the cell material 115 under study can be investigated separately. This individual investigation can be carried out simultaneously just be evaluating the recorded light intensities in a pixel wise manner. Thereby, each pixel of the first camera 171 should be assigned to a defined pixel of the second camera 172 in order to make a combined spatial resolving analysis for both measurement lights 151 and 152 possible.

It has to be mentioned that the sensitivity of the detection of the autofluorescence light 152 can be increased by using a pass filter 145, which effectively blocks visible light around 445 nm. In this case it can be guaranteed that all visible light respectively light around 445 nm, which may have been detected by the second camera 172, has indeed been generated by autofluorescence effects of NAD(P)H and not by light reaching the detector 172 for instance by unwanted scattering effects.

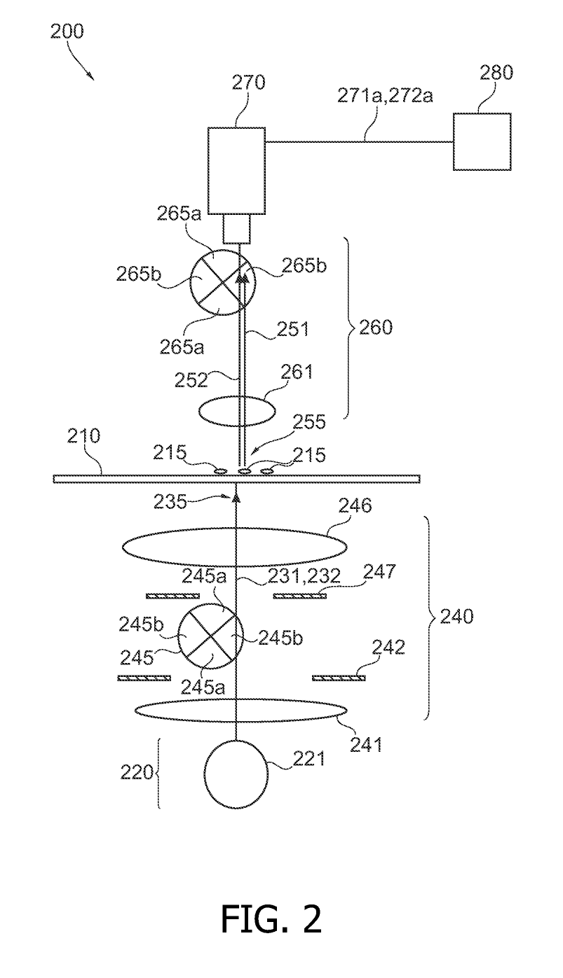

FIG. 2 shows a cell-analyzing device 200, which comprises a single broad-spectrum ultraviolet light source 221 and a common detector 270. The common detector 270 is used (a) for receiving a first measurement light 251 (260 nm) being indicative for the absorption of a first illumination light 231 (260 nm) caused by the amount of DNA within a cell material 215 and (b) for receiving a second measurement light 252 (445 nm), which is indicative for the strength of an autofluorescence signal of NAD(P)H caused by the excitation of NAD(P)H by means of a first illumination light 231 (340 nm).

The cell-analyzing device 200 comprises many components, which have already been explained in detail with reference to the embodiment shown in FIG. 1. Therefore, in order to avoid unnecessary repetitions, in the following predominately there will be described components, which components are different from the corresponding components of the device 100 or which components are not used in the cell-analyzing device 100.

By contrast to the cell-analyzing device 100, wherein a spatial separation between the first measurement light 151 and the second measurement light 152 is effected, the cell-analyzing device 200 uses a temporal separation in order to separate the first measurement light 251 from the second measurement light 252. This temporal separation is accomplished by two chopper wheels, a first chopper wheel 245 and a second chopper wheel 265.

The first chopper wheel 245 comprises an alternating sequence of spectral pass filters 245a and 245b. The pass filter 245a is transparent for radiation having a wavelength of around 260 nm whereas the pass filter 245b is transparent for radiation having a wavelength of around 340 nm.

The second chopper wheel 265 comprises an alternating sequence of spectral pass filters 265a and 265b. The pass filter 265a is transparent for radiation having a wavelength of around 260 nm whereas the pass filter 265b is transparent for radiation having a wavelength of around 445 nm.

The two chopper wheels 245 and 265 are operated in a synchronized manner such that within a first period of time the pass filter 245a is arranged within the illumination light beam 131, 132 and the pass filter 265a is arranged within the measurement light beam 151, 152. Correspondingly, within a second period of time the pass filter 245b is arranged within the illumination light beam 131, 132 and the pass filter 265b is arranged within the measurement light beam 151, 152. This means that (a) the DNA absorption measurements at a wavelength of 260 nm and (b) the autofluorescence measurements at 340 nm for excitation and 445 nm for de-excitation are carried out in a sequential manner.

It has to be mentioned that the embodiments shown in FIGS. 1 and 2 both have the advantage that only one single UV light source 121, 221 is necessary in order to do both UV cytometry absorption measurements and NAD(P)H excitations. By contrast to using two separate UV light sources the optical setup of the cell-analyzing devices 100, 200 is easier and the expenses for building the cell-analyzing devices 100, 200 are lower.

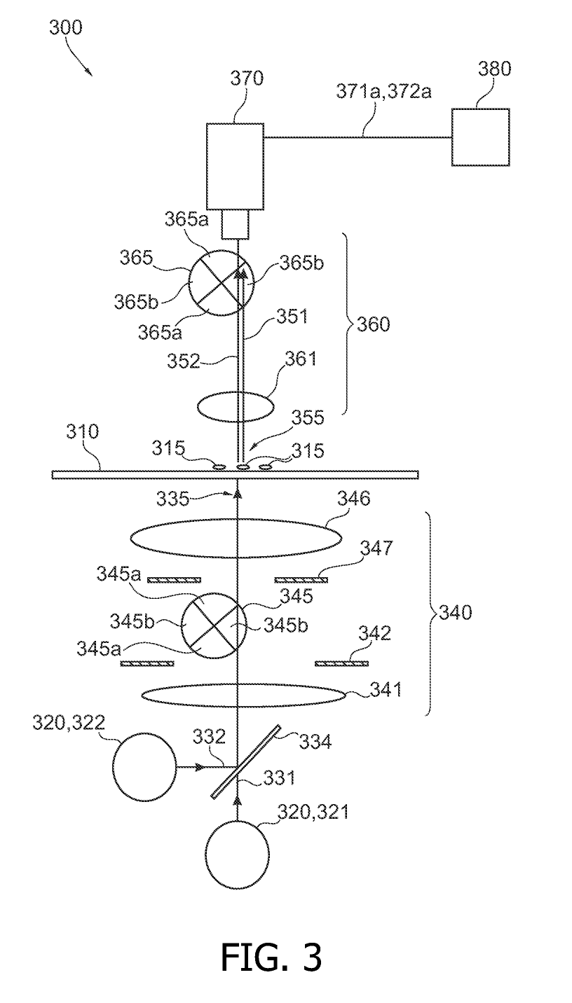

FIG. 3 shows a cell-analyzing device 300, which differs from the cell-analyzing device 200 shown in FIG. 2 by the provision of two UV light sources 321 and 322 instead of a single broad-spectrum light source 221. The first light source 321 is a deep ultraviolet light source such as a mercury lamp, which emits UV radiation including an intense 253 nm line. The second light source 322 may be a light emitting diode having an emission maximum preferably at 340 nm. The UV radiation emitted by these two UV light sources 321 and 322 is spatially combined by using a dichroic mirror 334, which is substantially transparent for 253 nm and reflective for 340 nm.

As has already been described above with reference to FIG. 2, a temporal separation between the first illumination light 331 and the second illumination light 332 is accomplished by a first chopper wheel 345. The separation between the first measurement light 351 and the second measurement light 352 is accomplished by a second chopper wheel 365, which is operable in a synchronized manner with respect to the first chopper wheel 345. For details about other components of the cell-analyzing device 300 reference is made to the previous description of the cell-analyzing devices 100 and 200.

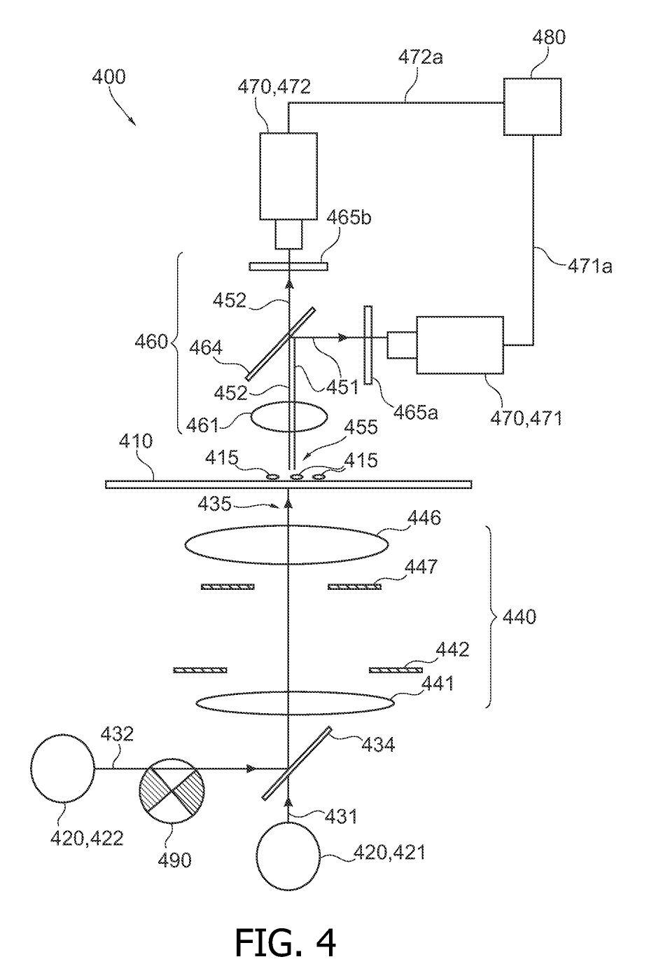

FIG. 4 shows a cell-analyzing device 400 for evaluating an autofluorescence lifetime of NAD(P)H being present within the cell 415 under study in combination with UV DNA absorptions measurements. The cell-analyzing device comprises two UV light sources 421 and 422 and two cameras 471 and 472.

The UV light source 421 is optically coupled to the first detector 471. The corresponding first illumination light beam 431 is transmitted through the dichroic mirror 434 and passes through the optic arrangement 440 before impinging onto the cell material 415. After a partial absorption caused predominately by the DNA within the nuclei of the cells 415, the remaining first measurement beam 431 enters the optic arrangement 460. At the dichroic mirror 464 the first measurement beam 431 is reflected and, after passing the pass filter 465a, the first measurement beam 431 is detected by the first camera 471.

In order to be able to measure the autofluorescence lifetime of NAD(P)H the intensity of the second illumination beam 432 has to be modulated in time. Preferably, the second illumination beam is discretely switched on and off such the temporal intensity hub is maximal. In the embodiment described in FIG. 4 this is achieved by a chopper device 490, which is positioned between the second light source 422 and the dichroic mirror 434.

The chopper device 490 comprises a segmented shutter wheel, which upon rotation repeatedly blocks the second illumination light 432. Thereby, a pulsed excitation of NAD(P)H is generated. Each pulsed excitation causes a subsequent temporal decay of the corresponding autofluorescence signal, which is received as the second measurement light beam 452 by the second camera 472. Since the excitation of the NAD(P)H is not accomplished with an infinite short excitation pulse but within a limited time span given by the rotation frequency and the spatial segmentation of the shutter wheel 490, it is easier to observe the average fluorescence lifetime of the excited NAD(P)H.

For details about other components of the cell-analyzing device 400 reference is made to the previous description of the cell-analyzing devices 100, 200 and 300, wherein corresponding components are described. As has already been indicated above, these components are denominated with reference signs, which are different from the corresponding reference signs only within the first digit.

It has to be mentioned that in order to observe the average fluorescence lifetime it is essential that at least the second camera 472 is capable of observing a time dependency of the intensity of the second measurement light 452. This time dependency can be used in combination with the value for the UV absorption caused by the DNA of the same cell material 415 for a reliable evaluation of the type of the cell material 415.

Further, it has to be mentioned that of course the invention is not limited to a combined measurement of DNA absorption and autofluorescence of NAD(P)H. The invention can also be realized with other components of human or animal cell. Depending on the spectral optical properties of these components also other wavelengths both for the first illumination light and for the second illumination light can be used. The same holds for filter elements, which can also be adapted to other spectral ranges both of the first measurement light and of the second measurement light.

All the described cell-analyzing devices 100, 200, 300, 400 for analyzing biological cell material 115, 215, 315, 415 can be applied in particular in hospitals or ambulatories during operations when a surgeon wants fast information on tissue malignancy when cutting out tumors. Furthermore, the described cell-analyzing devices 100, 200, 300, 400 can be used for cancer screening purposes.

It should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

In order to recapitulate the above described embodiments of the present invention one can state:

It is described a device 100 for analyzing biological cell material 115. The device 100 comprises a light source arrangement 120, which is adapted for directing a first 131 and a second illumination light 132 towards the cell material 115, wherein the first 131 and the second illumination light 132 comprises a first and a second spectral radiation component, respectively. The device 100 further comprises a detector arrangement 170, which is adapted for receiving a first measurement light 151 based on a first interaction of the first illumination light 131 with the cell material 115 and a second measurement light 152 based on a second interaction of the second illumination light 152 with the cell material 115. Further, the device 100 comprises an evaluation unit 180, which is coupled to the detector arrangement 170 and which is adapted to evaluate a first signal 171a and a second signal 171b being indicative for the first 151 and the second measurement light 151, respectively. The device 100 may be used for accomplishing ultraviolet DNA image cytometry in combination with autofluorescence measurements of NAD(P)H.

### LIST OF REFERENCE SIGNS

100 cell-analyzing device
110 carrier element, object holder
115 cell material
120 light source arrangement
121 broad-spectrum ultraviolet light source
131 first illumination light
132 second illumination light
135 common incidence beam path
140 optic arrangement
141 field lens
142 field iris
145 pass filter (260 nm and 340 nm)
146 condenser lens
147 condenser iris
151 first measurement light, transmitted light
152 second measurement light, fluorescence light
155 common exit beam path
160 optic arrangement
161 objective lens
164 dichroic mirror (reflective for 260 nm, transparent for 445 nm)
165a pass filter (260 nm)
165b pass filter (445 nm)
170 detector arrangement
171 first detector, first camera
171a first signal
172 second detector, second camera
172a second signal
180 evaluation unit
200 cell-analyzing device
210 carrier element, object holder
215 cell material
220 light source arrangement
221 broad-spectrum ultraviolet light source
231 first illumination light
232 second illumination light
235 common incidence beam path
240 optic arrangement
241 field lens
242 field iris
245 first chopper wheel
245a pass filter (260 nm)
245b pass filter (340 nm)
246 condenser lens
247 condenser iris
251 first measurement light, transmitted light
252 second measurement light, fluorescence light
255 common exit beam path
260 optic arrangement
261 objective lens
265 second chopper wheel
265a pass filter (260 nm)
265b pass filter (445 nm)
270 detector arrangement, common detector, common camera
271a first signal
272a second signal
280 evaluation unit
300 cell-analyzing device
310 carrier element, object holder
315 cell material
320 light source arrangement
321 first light source, deep ultraviolet light source; mercury lamp
322 second light source, 340 nm LED
331 first illumination light
332 second illumination light
334 dichroic mirror (transparent for 253 nm, reflective for 340 nm)
335 common incidence beam path
340 optic arrangement
341 field lens
342 field iris
345 first chopper wheel
345a pass filter (260 nm)
345b pass filter (340 nm)
346 condenser lens
347 condenser iris
351 first measurement light, transmitted light
352 second measurement light, fluorescence light
355 common exit beam path
360 optic arrangement
361 objective lens
365 second chopper wheel
365a pass filter (260 nm)
365b pass filter (445 nm)
370 detector arrangement, common detector, common camera
371a first signal
372a second signal
380 evaluation unit
400 cell-analyzing device
410 carrier element, object holder
415 cell material
420 light source arrangement
421 first light source, deep ultraviolet light source; mercury lamp
422 second light source, 340 nm LED
431 first illumination light
432 second illumination light
434 dichroic mirror (reflective for 260 nm, transparent for 340 nm
435 common incidence beam path
440 optic arrangement
441 field lens
442 field iris
446 condenser lens
447 condenser iris
451 first measurement light, transmitted light
452 second measurement light, fluorescence light
455 common exit beam path
460 optic arrangement
461 objective lens
464 dichroic mirror (reflective for 260 nm, transparent for 445 nm
465a pass filter (260 nm)
465b pass filter (445 nm)
470 detector arrangement
471 first detector, first camera
471a first signal
472 second detector, second camera
472a second signal
480 evaluation unit
490 light modulation device, chopper device

1. A device for analyzing biological cell material (115, 215, 315, 415), the device comprising:
a light source arrangement (120, 220, 320, 420), which is adapted for directing a first illumination light (131, 231, 331, 431) and a second illumination light (132, 232, 332, 432) towards the biological cell material (115, 215, 315, 415), wherein the first illumination light (131, 231, 331, 431) comprises a first spectral radiation component and the second illumination light (132, 232, 332, 432) comprises a second spectral radiation component,
a detector arrangement (170, 270, 370, 470), which is adapted for receiving
a first measurement light (151, 251, 351, 451), which is based on a first interaction of the first illumination light (131, 231, 331, 431) with the cell material (115, 215, 315, 415), and
a second measurement light (132, 232, 332, 432), which is based on a second interaction of the second illumination light (132, 232, 332, 432) with the cell material (115, 215, 315, 415), and,
an evaluation unit (180, 280, 380, 480), which is coupled the detector arrangement (170, 270, 370, 470) and which is adapted to evaluate
a first signal (171a, 271a, 371a, 471a) being indicative for the first measurement light (151, 251, 351, 451) and
a second signal (172a, 272a, 372a, 472a) being indicative for the second measurement light (132, 232, 332, 432).

2. The device according to claim 1, further comprising:
a carrier element (110, 210, 310, 410) for supporting the cell material (115, 215, 315, 415).

3. The device according to claim 1, wherein the first interaction is absorption and/or the second interaction is fluorescence.

4. The device according to claim 3, wherein
the first illumination light (131, 231, 331, 431) is adapted to interact with a first cell component of the biological cell material (115, 215, 315, 415) by means of the first interaction and/or
the second illumination light (132, 232, 332, 432) is adapted to interact with a second cell component of the biological cell material (115, 215, 315, 415) by means of the second interaction.

5. The device according to claim 4, wherein
the first cell component is DNA and/or
the second cell component is an enzyme being used for cell metabolism, in particular NAD(P)H.

6. The device according to claim 1, wherein
the first illumination light (131, 231, 331, 431) and the second illumination light (132, 232, 332, 432) is ultraviolet light comprising a wavelength in between 150 nm and 350 nm, preferably in between 200 nm and 300 nm and more preferably in between 230 nm and 270 nm.

7. The device according to claim 1, wherein
the second illumination light (132, 232, 332, 432) is ultraviolet light comprising a wavelength in between 280 nm and 400 nm, preferably in between 320 nm and 360 nm and more preferably in between 330 nm and 350 nm, and
the second measurement light (152, 252, 352, 452) is visible light comprising a wavelength in between 345 nm and 545 nm, preferably in between 400 nm and 490 nm and more preferably in between 430 nm and 460 nm.

8. The device according to claim 1, wherein
the light source arrangement (120, 220, 320, 420) comprises a broad-spectrum ultraviolet light source (121, 221).

9. The device according to claim 1, wherein
the light source arrangement (120, 220, 320, 420) comprises
a first light source (321, 421) generating the first illumination light (131, 231, 331, 431) and
a second light source (322, 422) generating the second illumination light (132, 232, 332, 432).

10. The device according to claim 1, further comprising
an optic arrangement (140, 240, 340, 440) for focusing the first illumination light (131, 231, 331, 431) and/or the second illumination light (132, 232, 332, 432) onto the biological cell material (115, 215, 315, 415).

11. The device according to claim 1, wherein
the detector arrangement (170, 270, 370, 470) comprises
a first detector (171, 471) for receiving the first measurement light (151, 251, 351, 451) and
a second detector (172, 472) for receiving the second measurement light (132, 232, 332, 432).

12. The device according to claim 1, wherein
the detector arrangement (170, 270, 370, 470) comprises
a common detector (270, 370) for receiving both the first measurement light (151, 251, 351, 451) and the second measurement light (132, 232, 332, 432), and wherein
the device further comprises
a chopper device (245, 265, 345, 365) for letting pass the first measurement light (151, 251, 351, 451) and the second measurement light (132, 232, 332, 432) in an alternating manner.

13. The device according to claim 1, wherein
the first illumination light (131, 231, 331, 431) and the second illumination light (132, 232, 332, 432) impinge onto the biological cell material (115, 215, 315, 415) along a common incidence beam path (135, 235, 335, 435) and
the first measurement light (151, 251, 351, 451) and the second measurement light (132, 232, 332, 432) leave the biological cell material (115, 215, 315, 415) along a common exit beam path (155, 255, 355, 455), wherein
the common incidence beam path (135, 235, 335, 435) and the common exit beam path (155, 255, 355, 455) are collinear with respect to each other.

14. The device according to claim 3, wherein
the detector arrangement (470) is adapted to measure a time dependence of the second signal (472a) being indicative for the second measurement light (432) and
the evaluation unit (480) is adapted to evaluate the time dependence of the second signal (472a).

15. The device according to claim 14, further comprising
a light modulation device (490), which is adapted to modulate to intensity of the second illumination light (432) as a function of time.

16. A method for analyzing biological cell material (115, 215, 315, 415), the method comprising
directing a first illumination light (131, 231, 331, 431) comprising a first spectral radiation component from a light source arrangement (120, 220, 320, 420) towards the cell material (115, 215, 315, 415),
directing a second illumination light (132, 232, 332, 432) comprising a second spectral radiation component from the light source arrangement (120, 220, 320, 420) towards the cell material (115, 215, 315, 415),
receiving a first measurement light (151, 251, 351, 451), which is based on a first interaction of the first illumination light (131, 231, 331, 431) with the cell material (115, 215, 315, 415), by means of a detector arrangement (170, 270, 370, 470),
receiving a second measurement light (132, 232, 332, 432), which is based on a second interaction of the second illumination light (132, 232, 332, 432) with the cell material (115, 215, 315, 415), by means of the detector arrangement (170, 270, 370, 470),
evaluating a first signal being indicative for the first measurement light (151, 251, 351, 451), and
evaluating a second signal being indicative for the second measurement light (132, 232, 332, 432).
